(19) United States
(12) Patent Application Publication
(10) Pub. No.: US 2016/0128971 A1
(43) Pub. Date: May 12, 2016

(54) Nanoparticle Compositions

(71) Applicants: Vuong Trieu, Agoura Hills, CA (US); Kouros Motamed, Irvine, CA (US)

(72) Inventors: Vuong Trieu, Agoura Hills, CA (US); Kouros Motamed, Irvine, CA (US)

(21) Appl. No.: 14/245,997

(22) Filed: Apr. 4, 2014

Related U.S. Application Data
(63) Continuation of application No. 61/853,464, filed on Apr. 5, 2013.

(51) Int. Cl. A61K 31/337; A61K 31/7068; A61K 9/50; A61K 33/24

(52) U.S. Cl. CPC A61K 31/337; A61K 33/24; A61K 31/7068; A61K 9/5031

(57) ABSTRACT

The present invention relates to compositions and methods of formulating nanoparticle drugs for cancer treatment in particular for intravenous administration in particular nanoparticle formulations containing cytotoxic drugs for the treatment of cancer. The compositions may have properties which facilitate the release of drugs into the patient including being unstable in plasma/blood, having low AUC, low Cₘₐₓ, high Vd, CMC above theoretical Cₘₐₓ of the drug, high tumor/plasma AUC. The present invention also provides for methods of administration and compositions which are unstable after administration to a patient so that the cytotoxic drug may bind to endogenous proteins such as albumin and be delivered to tumors in the patient.

### CROSS-REFERENCE TO RELATED APPLICATIONS

This application claims the benefit of priority to U.S. Provisional Application No. 61/853,464 filed Apr. 5, 2013 which is incorporated by reference in its entirety.

### STATEMENT REGARDING FEDERALLY SPONSORED RESEARCH OR DEVELOPMENT

Not applicable.

### FIELD OF THE INVENTION

The present invention relates to compositions and methods of formulating nanoparticle drugs for cancer treatment in particular for intravenous administration in particular nanoparticle formulations containing cytotoxic drugs for the treatment of cancer. The compositions have properties which facilitate the release of drugs into the patient including being unstable in plasma/blood, having low AUC, low Cₘₐₓ, high Vd, CMC above theoretical Cₘₐₓ of the drug, high tumor/plasma AUC. The present invention also provides for methods of administration and compositions which are unstable after administration to a patient so that the cytotoxic drug may bind to endogenous proteins and be delivered to tumors in the patient.

### BACKGROUND OF THE INVENTION

Recent years have witnessed unprecedented growth of research and applications in the area of nanoscience and nanotechnology. There is increasing optimism that nanotechnology, as applied to medicine, will bring significant advances in the diagnosis and treatment of disease. Anticipated applications in medicine include drug delivery, both in vitro and in vivo diagnostics, nutraceuticals and production of improved biocompatible materials. Currently many substances are under investigation for drug delivery and more specifically for cancer therapy. Interestingly pharmaceutical sciences are using nanoparticles to reduce toxicity and side effects of drugs. Many drugs used to treat patients are administered by an intravenous route.

Abraxane® and Taxol® are chemotherapeutic drugs. Both drugs are used to treat solid tumours such as breast cancer and lung cancer. These cytotoxic medicines arrest the growth of cells in case of cancerous tissues. They essentially differ in the excipients they carry and their effectiveness. Paclitaxel is an antineoplastic drug used in chemotherapy. It is an alkaloid derived from plants and prevents microtubule formation in cells. The drug is solvent based and should be carefully administered since it is an irritant. The dosage and duration of administration of drug depends on the Body Mass Index. Side effects of Taxol include bone marrow suppression (primarily neutropenia), hair loss, arthralgias and myalgias, pain in the joints and muscles, peripheral neuropathy, nausea and vomiting, diarrhea, mouth sores, and hypersensitivity reaction, which can be dose limiting.

Abraxane® is paciltaxel formulated as albumin encapsulated nanoparticles. The Abraxane formulation is free of solvent—Cremophor—in Taxol®. The absence of solvent, allows the paclitaxel to bind to endogenous proteins and be transported by protein (ie. albumin) mediated transport mechanism. Protein receptors are common on the surface of tumor cells which facilitates the binding of drug molecule.

IG-001, also known as Genexol-PM or Paxus-PM, is a Cremophor-free novel nanoparticle formulation of paclitaxel. IG-001 is a polymer bound nanoparticle paclitaxel. Instead of using biological polymer (ie. albumin as in Abraxane) to encapsulate the paclitaxel, IG-001 uses diblock mPEG-PDLLA polymer. Biodegradable polymeric micelle-type or nanoparticle drug compositions, containing a water-soluble amphiphilic block copolymer having a hydrophilic poly(alkylene oxide) component and a hydrophobic biodegradable component, have been used to develop formulations in which a hydrophobic drug is physically trapped in the micelle. This micelle-type composition, enveloping a hydrophobic drug, can solubilize the hydrophobic drug in a hydrophilic environment to form a solution.

Nanomedicine is an emerging field of medicine in which drug treatments can be improved by formulating new delivery systems for drugs without using solvents found in traditional formulations. However, many challenges must be overcome if the application of nanotechnology is to realize the anticipated improved understanding of the patho-physiological basis of disease, bring more sophisticated diagnostic opportunities, and yield improved therapies. New compositions and criteria for formulating nanoparticles which can be used for intravenous administration with a variety of drug components are essential to the progress of nanomedicine. In addition, while there are many cytotoxic drug compositions that are useful in the treatment of various cancers, there exists a need for formulating cancer drug compositions which can be easily administered and achieve maximum clinical effectiveness with low toxicity.

### SUMMARY OF THE INVENTION

The present invention relates to novel compositions and methods of treatment of patients using nanoparticle formulated drugs used for intravenous administration where the properties of the nanoparticle been engineered to achieved desired properties including certain pharmacokinetic (pK) parameters. The formulations of the present invention may provide drugs entrained by nanoparticles which have properties which are counterintuitive when compared to traditional formulations. For example an effective nanoparticle formulation for intravenous administration of drugs for the treatment of patients might be unstable in plasma/blood, provide for rapid drug release, exhibit low Cₘₐₓ, AUC and high Vd; characteristics of rapid tissue penetration.

The present invention also relates to methods of treatment of cancer patients with nanoparticle formulated cancer treatment drugs including cytotoxic drugs where the properties of the nanoparticle been engineered to achieved desired properties including certain pharmacokinetic parameters. The formulations of the present invention may provide cytotoxic drug entrained by nanoparticles which have properties which are counterintuitive when compared to traditional formulations. For example an effective nanoparticle formulation for intravenous administration of cytotoxic drugs might be unstable in plasma/blood, provide for rapid drug release, exhibit low Cₘₐₓ, AUC and high Vd; characteristics of rapid tissue penetration.

The present invention relates to cytotoxic drug compositions comprising a cytotoxic drug entrained in a nanoparticle where the composition has a critical micelle concentration (CMC) higher than the theoretical Cₘₐₓ.

The present invention relates to a cytotoxic drug composition comprising a cytotoxic drug entrained in a where the composition has a CMC higher than the Cₘₐₓ and where the composition may be bound to and transported by endogenous proteins such as albumin in a mammal.

The present invention relates to cytotoxic drug compositions comprising one or more cytotoxic drugs encapsulated in a diblock copolymer wherein the composition is has a critical micelle concentration (CMC) higher than the theoretical Cₘₐₓ.

The present invention relates to a cytotoxic drug composition comprising one or more cytotoxic drugs encapsulated in a diblock copolymer where the composition has a CMC higher than the theoretical Cₘₐₓ and where the composition may be bound to and transported by endogenous proteins such as albumin in a mammal.

The present invention also related to cytotoxic drug compositions comprising one or more cytotoxic drugs entrained in a nanoparticle where the composition has a low AUC or Cₘₐₓ or high Vd such that the composition is bound to and transported by endogenous proteins such as albumin when administered to a human.

The present invention also related to cytotoxic drug compositions comprising one or more cytotoxic drugs encapsulated in a diblock copolymer where the composition has a low AUC or Cₘₐₓ or high Vd such that the composition is bound to and transported by endogenous proteins such as albumin when administered to a human.

The present invention also relates to methods of administering the cytotoxic compositions of the present invention as well as administering these compositions in combination with other active cancer treating agents such as cisplatin/carboplatin and gemcitabine.

The present invention also relates to methods of administering the cytotoxic compositions of the present invention to treat breast cancer or pancreatic cancer or lung cancer or bladder cancer or ovarian cancer.

The present invention also relates to methods of determining the optimal formulation for a cytotoxic drug containing nanoparticle for the treatment of cancer including determining the optimal Cₘₐₓ, CMC, AUC and Vd.

### BRIEF DESCRIPTION OF THE FIGURES

FIG. 1a. Paclitaxel release from various formulations

FIG. 1b. Paclitaxel release evaluated by Rapid Equilibrium Dialysis

FIG. 2a. Dose-proportionality graph for IG-001, IG-002, Taxol™ and Abraxane

FIG. 2b. Volume of distribution graph for IG-001, IG-002, Taxol, and Abraxane

FIG. 3. Plot of tumor growth inhibition ratio (T/C) for IG-001 versus T/C for Taxol for various tumor types

FIG. 4. Plot of tumor volume versus time for SKOV3 (ovarian cancer) when treated with IG-001, Taxol and control.

FIG. 5. Plot of tumor volume versus time for DLD-1 (colon cancer) when treated with IG-001, Taxol and control.

FIG. 6. Plot of tumor volume versus time for NIH H1299 (lung cancer) when treated with IG-001, Taxol and control.

FIG. 7. Plot of tumor volume versus time for AsPC-1 (pancreatic cancer) when treated with IG-001, Abraxane, gemcitabine and control.

FIG. 8. Plot of tumor volume versus time for PANC-1 (pancreatic cancer) when treated with IG-001 (20 mg/kg), IG-001 (50 mg/kg), Taxol (20 mg/kg), gemcitabine (140 mg/kg) and control.

FIG. 9. Plot of tumor volume versus time for PaCa-2 (early pancreatic cancer) when treated with IG-001 (25 mg/kg), IG-001 (40 mg/kg), IG-001 (60 mg/kg), Taxol (25 mg/kg), gemcitabine (140 mg/kg) and control.

FIG. 10. Dissolution profile of IG-001

### DETAILED DESCRIPTION OF THE INVENTION

The present invention relates to novel compositions for the treatment of patients using nanoparticle formulated drugs used for intravenous administration where the properties of the nanoparticle been engineered to achieved desired properties including certain pharmacokinetic (pK) parameters. The present invention relates to compositions and methods of formulating nanoparticle drugs for intravenous administration in particular nanoparticle formulations containing cytotoxic drugs for the treatment of cancer. The compositions may have properties which facilitate the release of drugs into the patient including being unstable in plasma/blood, having low AUC, low Cmax, high Vd, CMC above theoretical Cₘₐₓ of the drug, high tumor/plasma AUC. The present invention also provides for methods of administration and compositions which are unstable after administration to a patient so that the cytotoxic drug may bind to endogenous proteins such as albumin and be delivered to tumors in the patient.

Pharmacokinetics describes, quantitatively, the various steps of drug distribution in the body including the absorption of drugs, distribution of drugs to various organs and the elimination of drugs from the body. Various pharmacokinetic (pK) parameters include maximum observed plasma concentration (Cₘₐₓ), areas under the plasma concentration-time curve (AUCₗₐₛₜ and AUC_{inf}), areas under the first moment curve (AUMCₗₐₛₜ and AUMC_{inf}), time-to-maximum observed plasma concentration (Tₘₐₓ), half-life (T_{1/2}), the apparent terminal elimination rate constant (λ_{z}), and mean resident time (MRT).

Cₘₐₓ refers to the maximum concentration that a drug achieves in tested area after the drug has been administered. The Area Under the Curve (AUC) is a plot of concentration of drug in blood plasma against time. The area is computed from the time the drug is administered to the point where concentration in plasma is negligible. The Volume of Distribution (Vd) relates the amount of drug in the body to the measured concentration in the plasma. A large volume of distribution indicates that the drug distributes extensively into body tissues and fluids. Dose proportionality is also a common phrase used pharmacokinetics. Dose proportionality occurs when increases in the administered dose are accompanied by proportional increases in a measure of exposure like AUC or Cₘₐₓ. Thus an evaluation of dose proportionality usually includes exposure analysis of 3 or more doses to produce a graph. A discussion of various pharmacokinetic parameters and the methods of measuring them can be found in Clinical Pharmacokinetics and Pharmacodynamics: Concepts and Applications, M. Rowland and T. N. Tozer, (Lippincott, Williams & Wilkins, 2010).

Polymeric micelles and nanoparticles have been used in the delivery of various drugs. Micelle stability is influenced by various factors depending on the media environment including polymer concentration, molecular mass of the core-forming block, drug incorporation, other proteins and/or cells found in serum or blood. Stability of micelles depends on the polymer concentration. Polymer micelles have a critical micelle concentration (CMC) that is the lowest concentration of polymers to produce a micelle structure. Thus, micelles form when the concentration of the surfactant is greater than the critical micelle concentration and the temperature of the system is greater than the critical micelle temperature. Micelles can form spontaneously because of the balance between entropy an enthalpy. In aqueous systems, the hydrophobic effect is the driving force for micelle formulation and surfactant molecules assembling reduce the entropy. As the concentration of the lipid increases, the unfavorable entropy considerations from the hydrophobic end of the molecule prevail. At this point the lipid hydrocarbon chains of a portion of the lipids must be sequestered away from the water. Therefore, the lipid starts to form micelles. When surfactants are present above the CMC, they can act as emulsifiers that will allow a compound that is normally insoluble to dissolve. The CMC may be determined by a variety of methods including but not limited to: 1) spectroscopic measurements using a fluorescence probe, an absorbance dye 2 and other probes; 2) electrochemical measurement using electrophoresis or capillary electrophoresis; 3) surface tension measurements and contact angle measurement; 4) optical measurements using light scattering, optical fibers and refraction; 5) other methods such as ITC, chromatography, ultrasonic velocity and others. One method for determining CMC is by particle dissolution. Starting with a certain concentration (e.g. 5 mg/ml), the drug is serially diluted in a testing matrix (PBS, blood, plasma, etc.) and the size of the nanoparticle is determined by DLS. The concentration at which the nanoparticles disappear is the CMC.

It is commonly believed that effective intravenous nanoparticle formulations requires high blood/plasma level, stable nanoparticle (nanoparticle with critical micelles concentration below its Cₘₐₓ in blood/plasma) and slow release of drug, and would be effective in cancerous tumors. However, the compositions and methods of the present invention unexpected point to the opposite conclusion. Namely, effective nanoparticle formulations for intravenous administration of drugs, especially cytotoxic drugs are unstable in plasma/blood, provide for rapid drug release, exhibit low Cₘₐₓ, AUC and high Vd all of which are characteristics of rapid tissue penetration. The nanoparticle formulations of the present invention may be more effective or equally effective as conventional solvent based formulations at equal dosing.

The prior art teaches methods to prepare sustained release micelles in which polymers with very low CMC (<0.1 μg/ml) can be used for prolonging the circulation time before the micelle degrades. Upon intravenous injection, the micelles undergo dilution in the body. If the CMC of the micelles is high, the concentration of the polymer or surfactant falls below the CMC upon dilution and hence, the micelles dissociate. Therefore, the prior art teaches that a higher concentration of the polymer or surfactant has to be used to prepare the micelles or nanoparticles so that they withstand the dilution up to 5 L in the blood. However, the use of high concentrations might not be feasible due to toxicity related dose limitations. Therefore, the polymers are also selected for low CMC allowing for it to withstand dilution in blood. If the polymer or surfactant has a CMC lower than 0.1 μg/ml, concentrations such as 5 mg/ml may be used to prepare a micelle formulation in order to counter the dilution effects in the blood. A variety of polymers including diblock copolymers, triblock copolymers and graft copolymers have been synthesized for this purpose. Thus, the prior art teaches that the nanoparticles should be crafted to be stable even after intravenous administration. The compositions of the present invention provide for formulations in which the nanoparticles are less stable once administered such that the drug compound can be released from the nanoparticle. Release from the nanoparticle make the drug compound available to the endogenous proteins such as albumin delivery system. Nanoparticles of the present invention have CMC values which are higher than the Cₘₐₓ of the composition once delivered to a patient. In the nanoparticles of the present invention the CMC of the nanoparticles may be at least 10% higher than the expected Cₘₐₓ of the nanoparticle composition. In the nanoparticles of the present invention the CMC of the nanoparticles may be at least 20% higher or 25% higher or 30% higher or 35% higher or 40% higher or 45% higher or 50% higher or 55% higher or 60% higher or 65% higher or 70% higher or 75% higher or 80% higher or 85% higher or 90% higher or 95% higher or 100% higher or 125% higher or 150% higher or 175% higher or 200% higher or 500% than the expected Cₘₐₓ of the nanoparticle composition. In the nanoparticles of the present invention the CMC of the nanoparticles may be between about 10% higher to about 250% higher or about 10% higher to about 150% higher or about 10% higher to about 125% higher or about 10% higher to about 100% higher or from about 10% higher to about 90% higher or from about 10% higher to about 80% higher or from about 10% higher to about 70% higher or from about 10% higher to about 60% higher or from about 10% higher to about 50% higher or from about 10% higher to about 40% higher or from about 10% higher to about 30% higher or about 10% higher to about 20% higher or about 20% higher to about 125% higher or about 20% higher to about 100% higher or from about 20% higher to about 90% higher or from about 20% higher to about 80% higher or from about 20% higher to about 70% higher or from about 20% higher to about 60% higher or from about 20% higher to about 50% higher or from about 20% higher to about 40% higher or from about 50% higher to about 250% or from about 50% higher to about 125% higher or from about 50% higher to about 100% higher than the expected Cmax of the nanoparticle composition.

The nanoparticles of the present invention ideally release their contents in vivo but are stable in an iv bag, in an infusion solution or in a reconstitution vial. The nanoparticles of the present invention can be altered to accommodate the particular pK profile that is desirable for the drug to be delivered. The present invention relates to novel compositions and methods of treatment of patients using nanoparticle formulated drugs used for intravenous administration where the properties of the nanoparticle been engineered to achieved desired properties including one or more pharmacokinetic (pK) parameters.

The nanoparticles of the present invention may have low AUC and low Cₘₐₓ. In some embodiments the AUC of the nanoparticles of the present invention are at least 5% or at least 10% or at least 20% or at least 30% or at least 40% or at least 50% or at least 60% or at least 70% or at least 80% or at least 90% or at least 100% less than comparable solvent based formulations. In some embodiments the AUC of the nanoparticles of the present invention are between about 5% to about 100% or from about 5% to about 75% or from about 5% to about 50% or from about 5% to about 25% or from about 5% to about 10% or from about 10% to about 75% or from about 10% to about 50% or from about 10% to about 25% or from about 25% to about 100% or from about 25% to about 75% or from about 25% to about 50% less than comparable solvent based formulations.

In some embodiments the Cₘₐₓ (adjusted for dose and infusion rate) of the nanoparticles of the present invention are at least 5% or at least 10% or at least 20% or at least 30% or at least 40% or at least 50% or at least 60% or at least 70% or at least 80% or at least 90% or at least 100% less than comparable solvent based formulations. In some embodiments the AUC of the nanoparticles of the present invention are between about 5% to about 100% or from about 5% to about 75% or from about 5% to about 50% or from about 5% to about 25% or from about 5% to about 10% or from about 10% to about 75% or from about 10% to about 50% or from about 10% to about 25% or from about 25% to about 100% or from about 25% to about 75% or from about 25% to about 50% less than comparable solvent based formulations.

In some embodiments the Vd of the nanoparticles of the present invention the Vd of the nanoparticles may be at least 20% higher or 25% higher or 30% higher or 35% higher or 40% higher or 45% higher or 50% higher or 55% higher or 60% higher or 65% higher or 70% higher or 75% higher or 80% higher or 85% higher or 90% higher or 95% higher or 100% higher or 125% higher or 150% higher or 175% higher or 200% higher or 500% than the expected Vd of the solvent based composition.

In the nanoparticles of the present invention the CMC of the nanoparticles may be between about 10% higher to about 250% higher or about 10% higher to about 150% higher or about 10% higher to about 125% higher or about 10% higher to about 100% higher or from about 10% higher to about 90% higher or from about 10% higher to about 80% higher or from about 10% higher to about 70% higher or from about 10% higher to about 60% higher or from about 10% higher to about 50% higher or from about 10% higher to about 40% higher or from about 10% higher to about 30% higher or about 10% higher to about 20% higher or about 20% higher to about 125% higher or about 20% higher to about 100% higher or from about 20% higher to about 90% higher or from about 20% higher to about 80% higher or from about 20% higher to about 70% higher or from about 20% higher to about 60% higher or from about 20% higher to about 50% higher or from about 20% higher to about 40% higher or from about 50% higher to about 250% or from about 50% higher to about 125% higher or from about 50% higher to about 100% higher than the Cmax of the solvent based formulation.

The nanoparticles of the present invention also expected to increase the Overall Response Rate (ORR) of a given drug. ORR is defined as the proportion of patients whose best overall response is either complete response (CR) or partial response (PR) according to the standard called “Response Evaluation Criteria in Solid Tumors” (RECIST), and can be a measure of “effectiveness” of a drug. The compositions of the present invention are superior in ORR to currently existing formulations and compositions using the same cancer treatment drug.

Various formulations of nanoparticles are contemplated by the compositions of the present invention. Nanoparticles include but are not limited to dendrimers, polymer micelles, niosomes, nanogels, solid lipid nanoparticles, lipid nanostructured systems, cubosomes, liposomes, peptide nanotulules, metal colloids, carbon nanotubules, fullerenes, gold nanoparticles, gold nanoshells, silicon nanoparticles and magnetic colloids.

The nanoparticles of the present invention include colloidal dispersion systems which include macromolecule complexes, nanocapsules, microspheres, beads, and lipid-based systems including oil-in-water emulsions, micelles, mixed micelles, and liposomes. Liposomes, which are artificial membrane vesicles are useful as delivery vehicles in vivo. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidyl choline. Block polymers may be useful in formulating the nanoparticles of the present invention. One example is block copolymers with cyclodextrins which provide drug delivery as supramolecular polymeric micelles. This involves non-covalent interactions between a macromolecular polymer, which works as a host, and a small polymer molecule, which works as a guest. Triblock copolymer micelles are flower-like micelles can be formed with a triblock copolymer with small hydrophobic ends and a long hydrophilic midsection. When dissolved in water, such polymer molecules assemble to form flower-like micellar structure. These flower-like micelles can dissolve the drug in the hydrophobic core. Drug release was faster with crystalline PLA blocks than amorphous PLA blocks, possibly because crystalline PLA stacks together, leaving the drug largely at the periphery while amorphous PLA might better integrate/disperse the drug within the polymer matrix. Most micelle-forming polymers are first dissolved in organic solvent followed by addition to an aqueous medium to form micelles. The use of organic solvents can be avoided for some triblock copolymer micelles. Furthermore, through suitable selection of polymers, greater drug loading as well as sustained drug release can be achieved. Freeze-dried micelles may be easily redispersable. Unimolecular micelles may also provide a mechanism for release of drugs. The unimolecular micelle is made out of a polymer that has several hydrophilic and hydrophobic portions in itself and forms a single molecular micelle. Lipids and PEG-like hydrophilic polymers can be conjugated to form such unimolecular micelles. One such polymer is core(laur) PEG. Multiarm block copolymers can also be used to formulate micelles. For instance star-shaped or multiarmed micelles can be formed with an amphiphilic block copolymer with multiple hydrophilic blocks and a single hydrophobic block. These polymers can form micelles if the number of arms is high enough. One such polymer is H40-PLA-mPEG. Graft polymers have recently attracted significant attention in preparing micelles. Cellulose graft polymers can be used to form micelles. The cellulose portion of the polymer can be the hydrophilic part, with any hydrophobic segment conjugated to it to form an amphiphilic graft polymer.

Polymers have some degree of toxicity even if they are biocompatible. Therefore, there is a need to synthesize materials that are more biocompatible for the preparation of micelles and incorporation of drugs. Oligopeptides can be very useful amphiphilic molecules for the preparation of micelles. Hydrophobic residues, such as alanine, can be used to synthesize the hydrophobic block and hydrophilic residues like histidine or lysine can be used to synthesize the hydrophilic block. Such molecules can be used as amphiphilic molecules to formulate micelles. A combination of polymer and polyamino acid can form an amphiphilic polymer. PEG-polyglutamic acid copolymer was used to prepare micelles.

The polymeric micelle nanoparticle formulations includes amphiphilic block copolymer which may comprise a hydrophilic block (A) and a hydrophobic block (B) linked with each other in the form of A-B, A-B-A or B-A-B structure. Additionally, the amphiphilic block copolymer may form core-shell type polymeric micelles in its aqueous solution state, wherein the hydrophobic block forms the core and the hydrophilic block forms the shell.

In one embodiment, the hydrophilic block (A) of the amphiphilic block copolymer may be polyethylene glycol (PEG) or monomethoxypolyethylene glycol (mPEG). Particularly, it may be mPEG. The hydrophilic block (A) may have a weight average molecular weight of 500-20,000 daltons, specifically 1,000-5,000 daltons, and more specifically 1,000-2,500 daltons.

The hydrophobic block (B) of the amphiphilic block copolymer may be a water-insoluble, biodegradable polymer. In one embodiment, the hydrophobic block (B) may be polylactic acid (PLA) or poly(lactic-co-glycolic acid) (PLGA). In another embodiment, the hydrophobic block (B) may have a weight average molecular weight of 500-20,000 daltons, specifically 1,000-5,000 daltons, and more specifically 1,000-2,500 daltons. Hydroxyl end groups of the hydrophobic block (B) may be protected with fatty acid groups, and particular examples of the fatty acid groups include acetate, propionate, butyrate, stearate, palmitate groups, and the like. The amphiphilic block copolymer comprising the hydrophilic block (A) and the hydrophobic block (B) may be present in the composition in an amount of 20-98 wt %, specifically 65-98 wt %, and more specifically 80-98 wt % based on the total dry weight of the composition.

In another embodiment, the hydrophilic block (A) and the hydrophobic block (B) may be present in the amphiphilic block copolymer in such a ratio that the copolymer comprises 40-70 wt %, specifically 50-60 wt % of the hydrophilic block (A) based on the weight of the copolymer. When the hydrophilic block (A) is present in a proportion less than 40%, the polymer has undesirably low solubility to water, resulting in difficulty in forming micelles. On the other hand, when the hydrophilic block (A) is present in a proportion greater than 70%, the polymer becomes too hydrophilic to form stable polymeric micelles, and thus the composition may not be used as a composition for solubilizing taxane.

A preferred paclitaxel formulation is IG-001 (also referred to as Genexol-PM™, Cynviloq™) which is a Cremophor™-free, polymeric micelle formulation of paclitaxel. IG-001 comprises biodegradable di-block copolymer composed of methoxypoly(ethyleneglycol)-poly (lactide) to form nanoparticles with a paclitaxel-containing hydrophobic core, and a hydrophilic shell. The micellar composition may be made by dissolving an amphipathic co-polymer, monomethoxypolyethylene glycol-polylactide with an average molecular weight of 1766-2000 daltons at 80° C. in ethanol. Paclitaxel is added to the dissolved co-polymer and the solution cooled to about 50° C. where room temperature water is added. Anhydrous lactose may be added and dissolved. The solution may then be filtered and lyophilized. The amount of paclitaxel in the micelle formulation can be altered. Less or more paclitaxel will change the loading % and change the CMC and properties of the formulation. The size of the nanoparticles for IG-001 is a Gaussian distribution where the mean particle size is about 10 nm to about 50 nm.

Cytotoxic drugs entrained or encapsulated in the nanoparticles of the present invention may include but are not limited to carboplatin, cisplatin, cyclophoshaminde, doxorubicin, etoposide, fluoruracil, gemcitabine, irinotecan, methotrexate, topotecan, vincristine, vinblastine, docetaxel, paclitaxel, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel, epothilone, 17-AAG, or rapamycin.

Cancer types for which the methods of the present invention may be useful include but are not limited to bladder cancer, ovarian cancer, breast cancer, pancreatic cancer, liver cancer, non-small cell lung cancer (NSCLC) and other lung cancers.

Other intravenous drugs suitable for administration with the nanoparticles of the present invention include but are not limited to those for the treatment of infectious disease, cancer and proliferative disease.

### EXAMPLES

### Example 1

### Paclitaxel Release

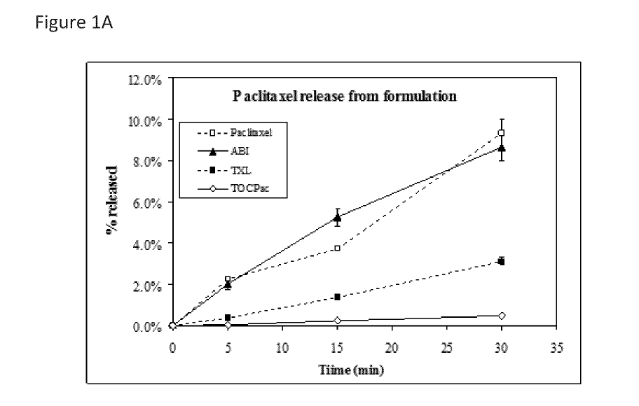

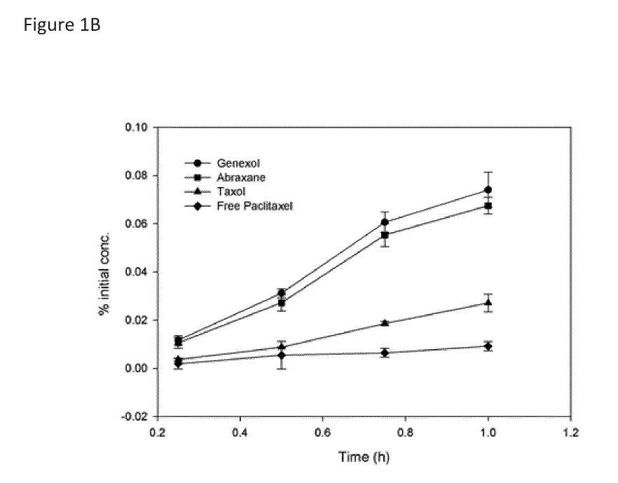

Paclitaxel release from each formulation was tested using equilibrium dialysis. Briefly, paclitaxel, IG-001, IG-002 (Tocosol-Pac), Taxol or reconstituted Abraxane (ABI) was added to one side of the well, and blank buffer to the other side. Samples were taken from the buffer side for the analysis of the appearance of free paclitaxel. The drug release profile from Abraxane appears similar to neat paclitaxel. Drug release is slowest for IG-002 (0.5% at 30 minutes, statistically significant versus the other three groups), followed by Taxol. Fast release was found for IG-001 and Abraxane. Results are shown in FIGS. 1a and 1b.

### Example 2

### Pharmacokinetics of Unstable Nanoparticles

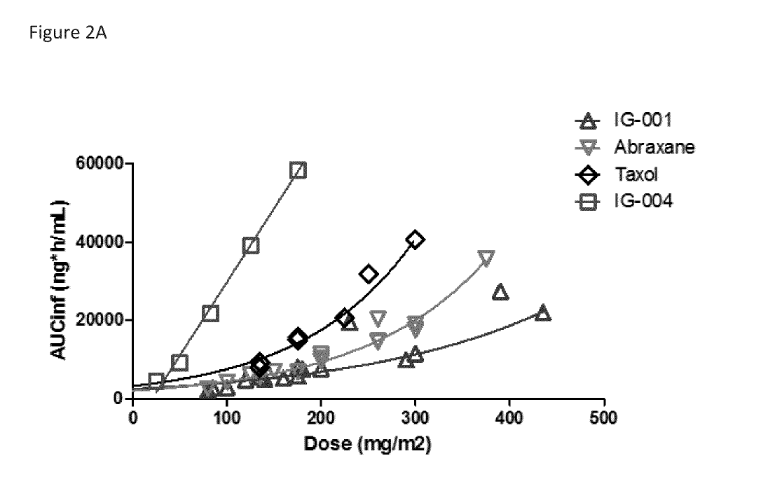

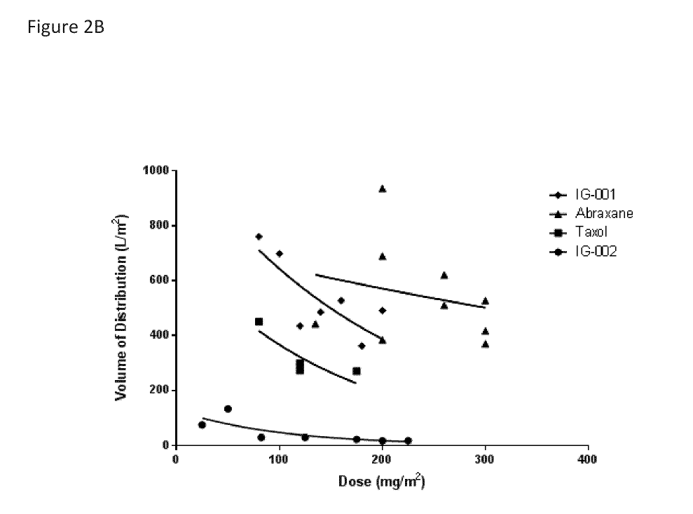

Clinical pharmacokinetics of IG-001 (Genexol-PM) was compared to Taxol and Abraxane and IG-002 (Tocosol). IG-001 range for PK dose-proportionality is the most expanded of the four paclitaxel formulations examined (Taxol, Abraxane, IG-001, IG-002) (FIG. 2a). Abraxane PK deviated from proportionality above 300 mg/m²; whereas IG-001 PK remained dose-proportional up to the highest dose of 435 mg/m². Additionally, the unstable nanoparticles IG-001 and Abraxane have lower AUC across all dose levels in comparison to the stable nanoparticle—IG-002/Tocosol.

Volume of distribution—Vd was higher for the unstable nanoparticles Abraxane and IG-001 versus stable nanoparticle (Tocosol/IG-002) or the solvent based paclitaxel formulation (Taxol). FIG. 2b.

### Example 3

### Tumor Growth Inhibition (T/C) for IG-001

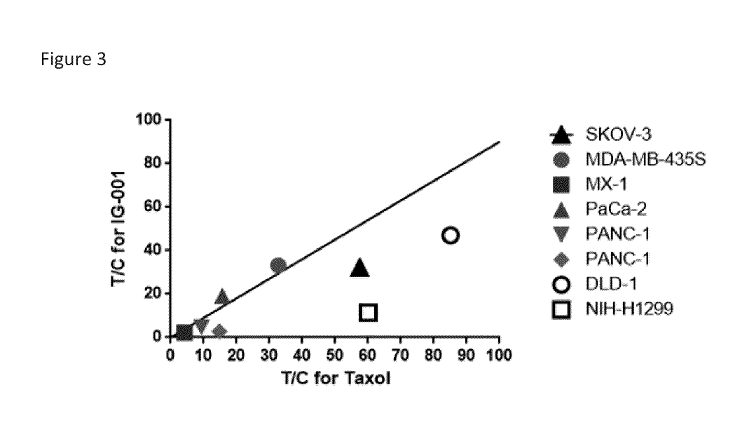

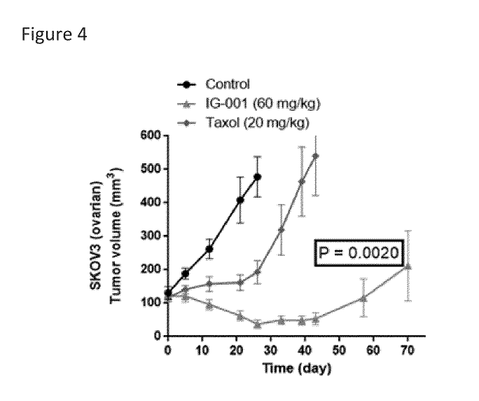

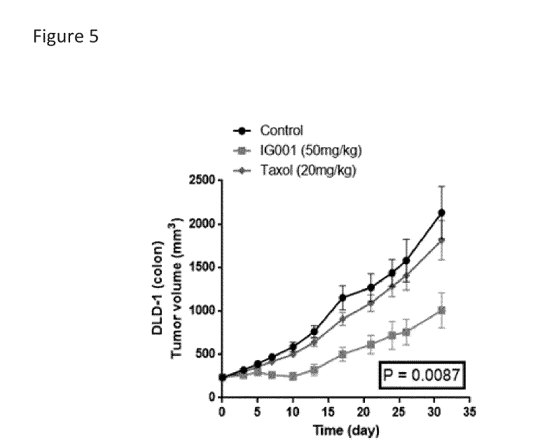

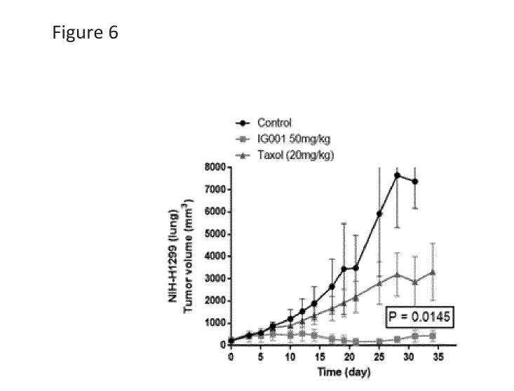

T/C using the AUC method was performed for a series of xenograft studies. The T/C of Taxol® was compared to T/C of IG-001, at equitoxic dose (20-25 mg/kg, qdx3, for Taxol® and 50-60 mg/kg, qdx3 for IG-001). Tumors resistant to Taxol® (SKOV3, DLD-1 and NIH-H1299) were more effectively treated with IG-001 (FIG. 3). Their T/C when treated with IG-001 was smaller than when treated with Taxol. ANOVA Statistic of Repeated Measurements was used to demonstrate statistical significant differences between Taxol and IG-001 for SKOV-3 (FIG. 4), DLD-1 (FIG. 5), and NIH H1299 (FIG. 6).

### Example 4

### Antitumor Activity of IG-001 Against Three Pancreatic Xenografts

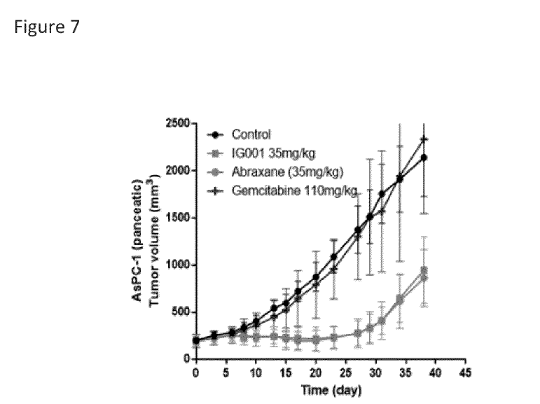

Male nude BALB/c mice (n=7/group) received a subcutaneous implantation of tumor fragments derived from the human pancreatic carcinoma cell line, AsPC-1. Tumors were allowed to reach 200 mm³ prior to initiation of intravenous (i.v.) treatments. Animals received treatments on Days 0, 3 & 6. Animals were monitored for 39 days (FIG. 7). Gemcitabine was dosed at q3dx12.

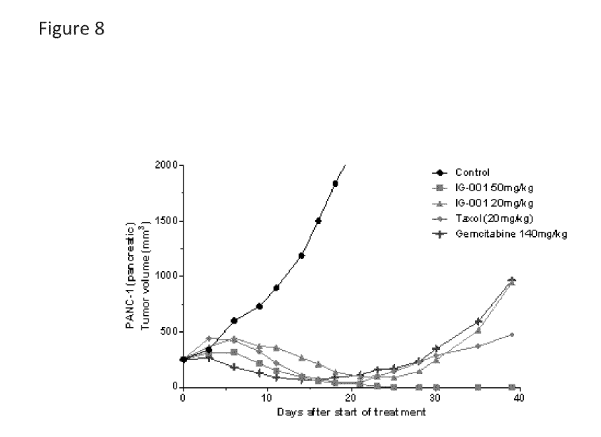

PANC-1 carcinoma: Groups of six tumor-bearing female athymic nude mice were treated with saline, Taxol® (q3dx3), Gemcitabine (q3dx12) or IG-001 (q3dx3) intravenously at the doses indicated. Tumor growth curves are shown for up to 39 days. The highest dose of IG-001 (50 mg/kg) resulted in complete remission of tumors vs. Taxol®. (FIG. 8)

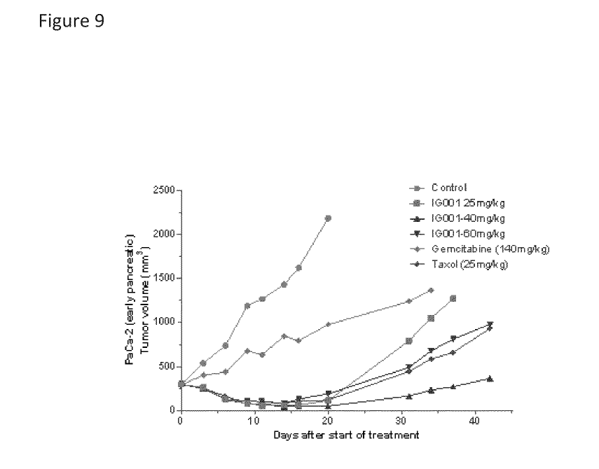

MIA PaCa-2 adenocarcinoma: Groups of seven tumor-bearing female athymic nude mice were treated with saline, Taxol® (q3dx3), Gemcitabine (q3dx12) or IG-001 (q3dx3) intravenously at the doses indicated. Tumor growth curves are shown for up to 45 days. IG-001 exhibited similar antitumor activity vs. Taxol®. (FIG. 9)

Of the three pancreatic tumor xenografts—the Taxol resistant PANC-1 was more effectively treated with IG-001 than Taxol.

### Example 5

### Dissolution in Serum

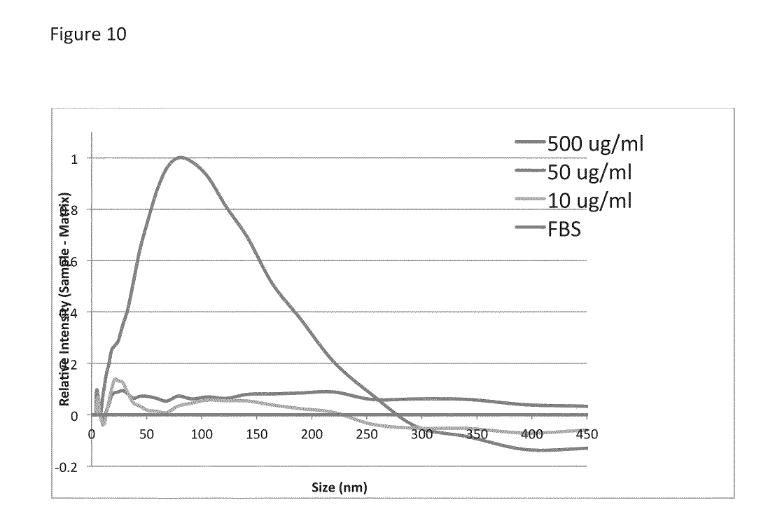

IG-001 (Genexol-PM) is a Cremophor-free, polymeric micelle formulation of paclitaxel utilizing biodegradable di-block copolymer composed of methoxy poly(ethylene glycol)-poly(lactide) to form nanoparticles with paclitaxel containing a hydrophobic core and a hydrophilic shell. IG-001 has a mean diameter of 25 nm with relatively low light scattering potential. IG-001 rapidly dissociates from intact nanoparticles upon dilution in serum at concentrations less than 50 ug/ml—higher than the Cₘₐₓ of IG-001—following a 3 hr infusion (FIG. 10). The CMC is higher than theoretical maximum drug level. Therefore, once administered, IG-001 readily gives up its paclitaxel cargo to endogenous proteins such as albumin for transport into the underlying tissues.

### Example 6

### Tumor Xenograft Distribution Data

Tumor distribution data for IG-001 in B16 and H460 tumor models were compared to available tumor distribution data for Abraxane. IG-001 and Abraxane® were characterized by low plasma AUC versus Taxol-solvent based traditionally formulated paclitaxel. Despite the low plasma AUC, IG-001 and Abraxane exhibited the same level of tumor accumulation—resulting in preferential accumulation in tumor versus plasma. IG-001 and Abraxane exhibited higher tumor accumulation than Taxol® relative to plasma level-3.2-3.6× and 2.0× advantage over Taxol®, for IG-001 and Abraxane®, respectively.

TABLE 1

| | | | AUC (tumor/ | AUC | | | | |
|---|---|---|---|---|---|---|---|---|
| | | Dose | μg/ml/ | (plasma/μg/ | Tumor | Plasma | | |
| Tumor | Agent | mg/kg | hr) | ml/hr) | (AUC/D) | (AUC/d) | Tumor/Plasma | Test/Taxol |
| B16 | IG-001 | 50.0 | 3714.28 | 77.0 | 74.5 | 1.5 | 48.5 | 1.9 |
| | IG-001 | 20.0 | 1290.8 | 16.3 | 64.5 | 0.8 | 79.4 | 3.2 |
| | IG-001 | 20.0 | 2139.41 | 95.0 | 107.0 | 4.3 | 25.2 | |
| | IG-001 | 50.0 | 273.07 | 61.3 | 5.5 | 1.2 | 4.5 | 2.3 |
| | IG-001 | 20.0 | 105.58 | 15.1 | 5.3 | 0.8 | 7.0 | 5.6 |
| | Taxol ® | 20.0 | 131.63 | 67.6 | 6.6 | 3.4 | 1.9 | |
| | Abraxane ® | 23.7 | 5869.0 | 1161.0 | 270.5 | 53.5 | 5.3 | 2.0 |
| | Taxol | 19.5 | 3716.0 | 1438.0 | 190.6 | 73.7 | 2.6 | |

*Abraxane ® data expressed as radioactivity. nCi · h/mL-source. EMEA.

### Example 7

### Phase II Data

Clinical efficacy of IG-001 (Genexol-PM) was compared to Taxol® and Abraxane® across three cancer indications (MBC, NSCLC, and Pancreatic Cancer). IG-001 was more active than historical Taxol®. Since pancreatic cancer is known to be poorly-perfused, IG-001 activity in this indication is consistent with it being able to penetrate poorly-perfused tumors.

TABLE 2
Metastatic Breast Cancer

| | | | SOLVENT BASED | | |
|---|---|---|---|---|---|
| | SOLVENT FREE | | Taxol ® | | |
| | IG-001 | | 2^{nd} line 6 | Taxotere ® | Taxotere ® |
| | First line | Abraxane ® | month | docetaxel | docetaxel |
| | 300 | First line | recur 175 | Anthra- | Alkylkating |
| | mg/m² | 260 mg/m² | mg/m² | pretreated | agent |
| | q3w | q3w | q3w | patients | pretreated |
| ORR | 59% | 42% | 28% | 28% | 45% |
| PFS | 9 Mo | 7 Mo | 4.2 Mo | 4.3 Mo | 6.5 Mo |
| OS | >20 Mo | 18 Mo | 11.7 Mo | 11.4 Mo | 14.7 Mo |

ORR—overall response rate
PFS—progression free survival
OS—Overall survival

TABLE 3
Advanced NSCLC

| | SOLVENT FREE | | | SOLVENT BASED | |
|---|---|---|---|---|---|
| | IG-001 | Abraxane ® | Abraxane ® | Taxol ® | Taxotere ®docetaxel |
| | First line | First line | First line | First line | First line |
| | 230-300 mg/m² | 260 mg/m² | 100 mg/m² | 250 mg/m² | 75 mg/m² |
| | q3w + CIS | q3w mono | Weekly + carbo | q3w + CIS | q3w + CIS |
| ORR | 38% | 15.5% | 33% | 23% | 31.6% |
| PFS | 5.8M | 6M | 6.3M | 4.9M | 4.9M |
| OS | 21.7M | 11.0M | 12.1M | 10.M | 10.9M |

CIS—Cisplatin

TABLE 4
Advanced Pancreatic Cancer

| | |
|---|---|
| | SOLVENT |
| SOLVENT FREE | BASED |

| | IG-001 | Abraxane ® | Taxol ® |
|---|---|---|---|
| | 1^{st} line | 2^{nd} line | 2^{nd} line |
| | 300-435 mg/m² | 100 mg/m² | 100 mg/m² |
| | q3wks | Wkly | Wkly |
| ORR | 6.7% | 5.3% | 10% |
| PFS | 3.2M | 1.7M | |
| OS | 6.5M | 7.8M | 6.7M |

Gem—gemcitabine

### Example 8

The PK parameters of PTX loaded PMs. This table is showing that all possible combinations of PKs in relation to Taxol can be obtained with different polymers/different nanoparticles. The composition of matter can be drawn from this table as well as from IG-001.

| Brand | Composition | Species, | Dose | Cₘₐₓ | AUC_{inf} | CL |
|---|---|---|---|---|---|---|
| name | of micelles | administration | (mg/kg) | (ug/ml) | (ugh/ml) | (l/h/kg) |
| Taxol | Cremophor | Patients, | 175 mg/m² | 3.31 ± 0.883uM | 11.8 ± 2.3 | 17.9 ± 3.64 L/h/m² |
| | PTX | infusion | | | (uM/h) | |
| PAX- | PTX-PDLLA₃₂₄₀- | | | | | |
| CEED | b-mPEG₂₀₀₀ | | | | | |
| Taxol | | Mice, i.v. | 20 | 94.08 ± 3.54 | 94.38 | 0.22 |
| GenexoI | | Mice, i.v. | 50 | | 82.83 ± 4.16 | 0.72 |
| GenexoI | PTX-PEO₂₀₀₀-b- | Patients | 175 mg/m² | 1.47 ± 0.208 | 5.74 ± 1.391 | 32.0 ± 8.8 L/h/m² |
| | PDLLA₁₇₅₀ | | | | | |
| Taxol | | Mice, i.v. | 50 | 12.50 | 91.3 | 0.5476 |
| NK 105 | PTX-PEO₈₀₀₀-b- | Mice, i.v. | 50 | 45.45 | 7862.3 | 0.0064 |
| | PPBA₁₂₀₀₀ | | | | | |
| NK 105 | PTX-PEO₈₀₀₀-b- | Patients, | 180 mg/m² | 45.6278 ± 8.6430 | 454.5 ± 119.1 | 0.4165 ± 0.1047 L/h/m² |
| | PPBA₁₂₀₀₀ | i.v. | | | | |
| Taxol | | Rats, i.v. | 3 | | 1007.9 ± 192.6 | 1.39 ± 0.39 L/h |
| | PTX-Pluronic | Rats, i.v. | 3 | | 2916.8 ± 873.6 | 0.65 ± 0.19 L/h |
| | P123 | | | | | |
| Taxol | | Rats, i.v. | 6 | | 4215.15 ± 2375.50 | 1.73 ± 0.74 |
| | PTX-P105 | Rats, i.v. | 6 | | 22191.48 ± 6093.14 | 0.29 ± 0.09 |
| | PTX-Pluronic | Rats, i.v. | 6 | | 20720.64 ± 12382.97 | 0.38 ± 0.22 |
| | P105/L101 | | | | | |
| Taxol | | Mice, i.v. | 10 | 0.0605 ± 0.0064 | 90.2 ± 24.5 | 2.7 ± 0.3 ml/h |
| | PTX-PEO₄₃ | Mice, i.v. | 10 | 0.0681 ± 0.0038 | 110.0 ± 29.1 | 1.3 ± 0.8 ml/h |
| | PPO₁₅PEO₄₃ | | | | | |
| Taxol | | Mice, oral | 10 | ND | ND | ND |
| | PTX-PEO₄₃ | Mice, oral | 10 | 19.2 ± 3.6 | 99.2 ± 17.3 | 1.1 ± 0.3 |
| | PPO₁₅PEO₄₃ | | | | | |
| Taxol | | Rats, i.v. | 5 | | 2.49250 ± 0.32259 | 1.87497 ± 0.32138 |
| | PTX-mPEG₅₀₀₀- | Rats, i.v. | 5 | | 2.53708 ± 0.36438 | 1.93140 ± 0.31828 |
| | PLA₃₂₅₀ | | | | | |
| | PTX-mPEG₅₀₀₀- | Rats, i.v. | 5 | | 2.35153 ± 0.25455 | 2.06560 ± 0.23249 |
| | PLA₃₂₅₀/Pluronic | | | | | |
| | L61 | | | | | |
| Taxol | | Rats, i.v. | 7 | | 12.84 | 0.5836 ± 0.0759 |
| | PTX-OSC | Rats, i.v. | 7 | | 3.83 ± 0.52 | 1.9083 ± 0.2688 |
| | PTX- | Rats, i.v. | 7 | | 11.31 ± 2.49 | 0.6702 ± 0.1528 |
| | mPEGOSC₂₀₀₀ | | | | | |
| Taxol | | Rats, i.v. | 4 | 4.8 ± 2.59 | 22.00 ± 6.40 | |
| | PTX-mPEG₂₀₀₀- | Rats, i.v. | 4 | 1.60 ± 2.79 | 8.43 ± 2.38 | |
| | PCL₂₀₀₀ | | | | | |

| | | | | Drug | |
|---|---|---|---|---|---|
| Brand | Composition | Species, | T_{1/2β} | loading | Size |
| name | of micelles | administration | (h) | (%) | (nm) |
| Taxol | Cremophor | Patients, | 6.96 ± 1.63 | | |
| | PTX | infusion | | | |
| PAX- | PTX-PDLLA₃₂₄₀- | | | 25 | |
| CEED | b-mPEG₂₀₀₀ | | | | |
| Taxol | | Mice, i.v. | 0.34 | | |
| GenexoI | | Mice, i.v. | 0.21 | | |
| GenexoI | PTX-PEO₂₀₀₀-b- | Patients | 12.5 ± 2.3 | | |
| | PDLLA₁₇₅₀ | | | | |
| Taxol | | Mice, i.v. | 0.98 | | |
| NK 105 | PTX-PEO₈₀₀₀-b- | Mice, i.v. | 5.99 | 23 | 85 |
| | PPBA₁₂₀₀₀ | | | | |
| NK 105 | PTX-PEO₈₀₀₀-b- | Patients, | 10.6 ± 1.3 | 23 | 85 |
| | PPBA₁₂₀₀₀ | i.v. | | | |
| Taxol | | Rats, i.v. | 2.50 ± 0.63 | | |
| | PTX-Pluronic | Rats, i.v. | 5.85 ± 1.52 | | 20.8 ± 2.9 |
| | P123 | | | | |
| Taxol | | Rats, i.v. | 1.27 ± 0.23 | | |
| | PTX-P105 | Rats, i.v. | 6.20 ± 2.01 | 1.0 | 23.9 ± 3.0 |
| | PTX-Pluronic | Rats, i.v. | 6.99 ± 4.12 | 1.7 | 185.3 ± 21.0 |
| | P105/L101 | | | | |
| Taxol | | Mice, i.v. | 2.27 ± 0.5 | | |
| | PTX-PEO₄₃ | Mice, i.v. | 2.2 ± 0.1 | 30 | 180.0 ± 5 |
| | PPO₁₅PEO₄₃ | | | | |
| Taxol | | Mice, oral | ND | | |
| | PTX-PEO₄₃ | Mice, oral | 0.1 ± 0 | 30 | 180.0 ± 5 |
| | PPO₁₅PEO₄₃ | | | | |
| Taxol | | Rats, i.v. | | | |
| | PTX-mPEG₅₀₀₀- | Rats, i.v. | | 13.37 ± 0.23 | 47.79 ± 0.86 |
| | PLA₃₂₅₀ | | | | |
| | PTX-mPEG₅₀₀₀- | Rats, i.v. | | 11.42 ± 0.04 | 72.2 ± 0.04 |
| | PLA₃₂₅₀/Pluronic | | | | |
| | L61 | | | | |
| Taxol | | Rats, i.v. | 1.695 ± 0.231 | | |
| | PTX-OSC | Rats, i.v. | 2.501 ± 0.109 | 60.91 | 200.8 |
| | PTX- | Rats, i.v. | 2.718 ± 0.125 | 41.1 ± 2.0 | 104.3 ± 5.8 |
| | mPEGOSC₂₀₀₀ | | | | |
| Taxol | | Rats, i.v. | | | |
| | PTX-mPEG₂₀₀₀- | Rats, i.v. | | 8.1 ± 0.3 | 28.6 ± 0.1 |
| | PCL₂₀₀₀ | | | | |

Cₘₐₓ maximum drug concentration;
AUC_{inf} area under the plasma concentration curve from 0 h to infinity;
CL, total clearance;
T_{1/2β,} half-life in the β(elimination) phase

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Further advantages of the present immunological compositions and adjuvants of the present invention can be achieved by those skilled in the art based upon the embodiments described herein and are thus specifically within the scope of the present invention.

1-52. (canceled)

53. A cytotoxic drug composition comprising a cytotoxic drug encapsulated in a nanoparticle wherein the composition has a critical micelle concentration (CMC) which is higher than the Cₘₐₓ when delivered intravenously to a human body or animals.

54. The composition of claim 53 wherein the nanoparticle is comprised of a diblock co-polymer.

55. The composition of claim 54 wherein the drug is paclitaxel.

56. The composition of claim 55 wherein the CMC of the composition is higher than the Cₘₐₓ by at least 5%.

57. The composition of claim 55 wherein the CMC of the composition is higher than the Cₘₐₓ by at least 10%.

58. The composition of claim 55 wherein the CMC of the composition is higher than the Cₘₐₓ by at least 25%.

59. The composition of claim 55 wherein the CMC of the composition is higher than the Cₘₐₓ by at least 50%.

60. The composition of claim 55 wherein the nanoparticle is comprised of a diblock copolymer.

61. The composition of claim 55 wherein the paclitaxel composition is IG-001.

62. A method of treating a patient with cancer by administering the composition of claim 55.

63. The method of claim 62 wherein the cancer is breast cancer

64. The method of claim 62 wherein the cancer is lung cancer.

65. The method of claim 62 wherein the cancer is pancreatic cancer

66. A method of treating a patient with cancer by administering the composition of claim 61 in combination with another active agent.

67. The method of claim 66 wherein the agent is cisplatin

68. The method of claim 66 wherein the agent is gemcitabine.

69-92. (canceled)
